(19) United States
(12) Patent Application Publication
(10) Pub. No.: US 2005/0250177 A1
(43) Pub. Date: Nov. 10, 2005

(54) Preparation, characterization and quantification of active fractions that promote cell growth and recombinant-protein expression

(76) Inventors: Faustinus K. Yeboah, Longueuil (CA); Barbara Jardin, Montreal (CA); Yasuo Konishi, Kirkland (CA); Amine A. Kamen, Montreal (CA); Tin Kam Kathy Wong, Singapore (SG); Soo Young Stanford Lee, Horsham, PA (US)

Correspondence Address: Ira S. Dorman, Suite 200, 330 Roberts Street, East Hartford, CT, 06108, US

(21) Appl. No.: 10/637,017

(22) Filed: Aug. 7, 2003

Related U.S. Application Data
(60) Provisional application No. 60/401,802, filed on Aug. 8, 2002.

(51) Int. Cl. C12P 21/06; C12N 5/02

(52) U.S. Cl. 435/68.1; 435/404

(57) ABSTRACT

A process for preparing an active animal cell-growth-enhancing fraction of a hydrolysate of plant tissue, animal tissue or microorganism ultrafiltrate material, especially a fraction of a yeastolate ultrafiltrate, comprising forming a precipitate from said fraction with a water-soluble solvent is disclosed. Such solvents as alkanols, alkyl sulfoxides, ketones or alkyl nitriles, especially lower (C₁-C₅) alkanols, particularly ethanol, can be used. The invention also relates to an active animal cell-growth-enhancing fraction of a hydrolysate of plant tissue, animal tissue or microorganism ultrafiltrate material, especially a fraction of a yeastolate ultrafiltrate, substantially free of aromatic and methyl group bearing compounds which has improved cell-growth-enhancing properties.

### BACKGROUND OF THE INVENTION

Yeast extracts from Saccharomyces cerevisiae (baker's yeast) have been traditionally used in animal cell cultures as a substitute to fetal bovine serum. Yeastolate ultrafiltrate (10 kDa cut-off) is a key component in formulating serum-free medium for insect cell cultures. Also, it is a key element in formulating cocktails that allow high cell density cultures and fed-batch operations. Yeastolate ultrafiltrate is a product derived from natural sources and as such many yeastolate preparations show significant variability. In fact, very few lots are identified as useful for insect cell culture. Identification of successful lots is only possible after a long process of cell growth and protein production testing.

As the activity of yeastolate is reproducible within the same lot, purchasing and using the same lot of yeastolate is a practical solution to overcome the lot dependence problem.

Current problems using yeastolate include:
(1) Significant lot-to-lot variability, which is not compatible with a GNP approach, required for expression of therapeutic proteins.
(2) Tedious screening method is required to find successful yeastolate lots.
(3) There are no defined media and this compromises rational research approaches.
(4) Some components of the yeastolate are toxic to the insect cells when used in serum-free conditions.

There is therefore a need for a component of a serum-free cell medium which is consistent in quality with very little, if any, batch-to-batch variability and is non-toxic to cells.

### SUMMARY OF THE INVENTION

According to one aspect of the invention, we have developed a robust method to prepare active fraction from yeastolate ultrafiltrate that promotes cell growth and protein production of insect cells.

According to another aspect of the invention, we have characterized the active fraction by analytical methods such as HPLC and NMR.

According to yet another aspect of the invention, we have developed a rapid method to finger print the active fraction in each lot of yeastolate ultrafiltrate.

According to a further aspect of the invention, a one step preparation of active fraction from yeastolate ultrafiltrate that promotes cell growth and protein production of insect cells is provided.

According to yet a further aspect of the invention, characterization of the active components semi-purified from yeastolate ultrafiltrate by ¹H- and ¹³C-NMR and HPLC, is provided.

According to another aspect of the invention, quantification of the major component in the active fraction from yeastolate ultrafiltrate by ¹H-NMR is provided.

The invention particularly provides a process for preparing an active animal cell-growth-enhancing fraction of a hydrolysate of plant tissue, animal tissue or microorganism ultrafiltrate material which process comprises forming a precipitate of said fraction from an aqueous solution of said hydrolysate of said plant, animal or microorganism ultrafiltrate with a water-miscible solvent. Preferably hydrolysate of plant tissue, animal tissue or microorganism ultrafiltrate material comprises a yeastolate ultrafiltrate. The process may additionally comprise separating and drying said precipitate and/or fractionating said precipitate and selecting high activity fractions. Preferably the water-miscible organic solvent is selected from the group consisting of alkanols, alkyl sulfoxides, ketones and alkyl nitrites, particularly a water-miscible solvent selected from the group consisting of an alcohol of the formula CH₃—(CH₂)ₙ—OH (n=1 to 4), an alkyl sulfoxide of the formula CH₃—(CH₂)ₙ—SO—(CH₂)ₘ—CH (n=0 to 2, m=0 to 2), an alkyl nitrite of the formula CH₃—(CH₂)ₙ—CN (n 0 to 2) and a ketone of the formula CH₃—(CH₂)ₙ—CO—(CH₂)ₙ—CH₃ (n=0 to 2, m=0 to 2). In a preferred embodiment the water-miscible solvent is a C₁-C₅ alcohol such as ethanol. In a particular embodiment of the invention the precipitate is formed with an ethanol concentration of about 65%. The invention also relates to an active animal cell-growth-enhancing fraction of a plant tissue, animal tissue or microorganism ultrafiltrate material substantially free of aromatic group bearing compounds prepared according to the process of the invention.

### BRIEF DESCRIPTION OF THE DRAWINGS

FIG. 1 shows reversed phase HPLC chromatograms of the yeastolate ultrafiltrate and the 80% EtOH precipitate of the yeastolate ultrafiltrate monitored at 215 nm.

FIG. 2 shows the growth of High-Five insect cells in the presence of various growth-promoting medium. The growth-promoting medium was reconstituted by replacing the yeastolate ultrafiltrate with double (PPT [2×]) or triple (PPT [3×]) equivalent amount of the 75% EtOH precipitate, assuming 100% recovery in EtOH fractionation. Single equivalent of the supernatant and the precipitate (1×SOL+1×PPT) was also added to reconstitute the yeastolate ultrafiltrate. The positive and negative control experiments were performed in the presence (Y total) and absence (0) of the yeastolate ultrafiltrate, respectively. A dose response effect is observed. An increase of growth from 60-71% to 104-113% is obtained by increasing the amount of the precipitate from 1 to 3 equivalent.

FIG. 3 is a graph illustrating the growth of High-Five insect cells in the presence single, double, and triple equivalent amounts of 60, 70, 75, 80, and 90% EtOH precipitates (PPt) and supernatants (Sol). The results show the average and standard deviations of the data of 6 different yeastolate lots in 96-well microplate. The data are normalized by taking positive control as 100% activity and the negative control as 0% activity.

FIG. 4 shows the growth of High-Five insect cells in the presence of single equivalent amount of the 80% EtOH precipitate or the supernatant in shake flask. The growth curve with the yeastolate ultrafiltrate is also shown as positive control. All data are averaged for 6 yeastolate lots with error bars of their standard deviations.

FIG. 5 shows the growth of Human Embryo Kidney 293 cells HEK-293 in the presence of the yeastolate ultrafiltrate. The cell growth was monitored by the green fluorescent protein (GFP) fluorescence at 3, 4, 5, 6, and 7^{th} days in culture as indicated by the color coded bars. Addition effects of yeastolate at half (½×) and standard concentration (1×) from three different lots are compared in duplicate or triplicate are compared to similar additions of bovine calf serum (BCS). The basal medium hybridoma serum free medium (HSFM) (Gibco/Invitrogen) is used as a negative control.

FIG. 6 illustrates the activity of each prep-HPLC fraction of the 75% EtOH precipitate in producing GFP by High-Five insect cells. The data at the fraction 0 comprise the positive control with the yeastolate ultrafiltrate.

FIG. 7 shows the ¹H-NMR spectra of (A) the active HPLC fraction of the 80% EtOH precipitate, of (B) 80% EtOH precipitate, and of (C) the yeastolate ultrafiltrate in D₂O. A comparison of the spectra shows that ethanol precipitation removes aromatic compounds from 4 the yeastolate. Compounds bearing the methyl groups between 0.6 and 1 ppm are also removed by 80% ethanol precipitation. HPLC purification eliminates residual aromatic compounds and compounds baring the methyl group(s) between 1 and 1.2 ppm. The relative intensities of peaks between 2.3 and 3.0 ppm are also enhanced by HPLC purification, indication that the active component(s) of yeastolate are non-aromatic compounds and contain protons with chemical shifts between 2.3 and 3.0 ppm.

FIG. 8 shows the UV/VIS absorption spectrum of the active RP-HPLC fraction of the 80% EtOH precipitate in water.

FIG. 9 shows the ¹³C-NMR spectrum of the active RP-HPLC fraction of 80% EtOH precipitate in D₂O. The NMR spectrum shows that fraction 6 contains at least 2 components. The molar ration of the major and the minor components is 2-3 to 1.

FIG. 10 shows the proton-proton COSY spectrum of the active RP-HPLC fraction of the 80% EtOH precipitate in D₂O. The off-diagonal peaks show the interactions between the protons connected to adjacent carbon atoms.

FIG. 11 shows the proton-proton TOCSY spectrum of the active RP-HPLC fraction of the 80% EtOH precipitate in D₂O. The off-diagonal peaks show both short- and long-distance interactions between protons within the same molecule.

FIG. 12 shows the HMQC spectrum of the active RP-HPLC fraction of the 80% EtOH precipitate in D₂O. The spectrum shows the carbon proton connections within the same molecule.

FIG. 13 emphasizes the peaks of the ¹H-NMR spectra of FIG. 7 that are enhanced by the 80% EtOH precipitation and the subsequent HPLC fractionation. Quantification of the enhanced proton peaks can be used as quality control of the growth promoting factor in various preparations of growth promoting media from the yeastolate.

FIG. 14 shows the growth curve of Sf9 cells in IPL-41 medium supplemented with or without yeastolate UF or its fractions.

FIG. 15 shows the effect of yeastolate UF or its fraction supplements on promoting the cell growth

FIG. 16 shows the dose response of yeastolate UF supplement on cell growth.

FIG. 17 shows the dose response of fraction 1 supplement on cell growth

FIG. 18 shows the influence of yeastolate UF or fraction 1 on the increase of cell counts.

FIGS. 19-23 illustrate characteristics of large scale preparation products of the invention.

FIG. 19. The profiles of ¹H-NMR spectra of (A) fraction 1 and of (B) yeastolate ultrafiltrate (solvent D₂O, temperature 25° C., instrument Bruker-DRX-500 MHz).

FIG. 20. The profile of ¹³C-NMR spectrum of fraction 1 (solvent D₂O, temperature 25° C., instrument Bruker-DRX-500 MHz).

FIG. 21. The ¹H-¹H COSY spectrum of fraction 1 (solvent D₂O, temperature 25° C., instrument Bruker-DRX-500 MHz).

FIG. 22. The ¹H-¹H TOCSY spectrum of fraction 1 (solvent D₂O, temperature 25° C., instrument Bruker-DRX-500 MHz).

FIG. 23. The HMQC spectrum of fraction 1 (solvent D₂O, temperature 25° C., instrument Bruker-DRX-500 MHz).

FIG. 24 shows fractionation results of 50× yeastolate UF by ethanol sequential precipitation.

Table 1 shows the yield of 60-90% EtOH precipitates.

Table 2 lists the peaks in H-NMR and C-13 NMR spectra of the active HPLC fraction of the 80% EtOH precipitate.

### DETAILED DESCRIPTION OF THE INVENTION

Serum-free commercial media are available for the cultivation of insect cells. The success of serum elimination is largely contributed by the supplementation of lipid emulsion and protein hydrolysates such as lactalbumin, tryptose phosphate broth, casein, and yeastolate. Though widely available and highly optimized, these media are expensive and suffer from batch to batch variation because of their pseudo-defined nature. The other major disadvantage of using these media is that their formulation is proprietary, making it difficult for process alteration and research. Complications are also introduced when products produced using these media reach the downstream processing end. This prompted several groups to develop in-house low cost media for both Sf and Tn cells. Again, undefined components such as Hy-Soy, Primatone RL and Yeastolate are needed as supplements in these media to match cell and product yields close to that in serum containing media. These low cost media are particularly useful for the production of inexpensive products such as animal vaccines and biopesticides, which do not require stringent purification processes. As the potential of using baculovirus expression systems (BEVS) increases for therapeutic protein production, the need for a defined medium also increases. The exact component(s) responsible for cell and product yield enhancement from protein hydrolysates has not yet been identified.

A One-Step Preparation of Active Fraction from Yeastolate Ultrafiltrate.

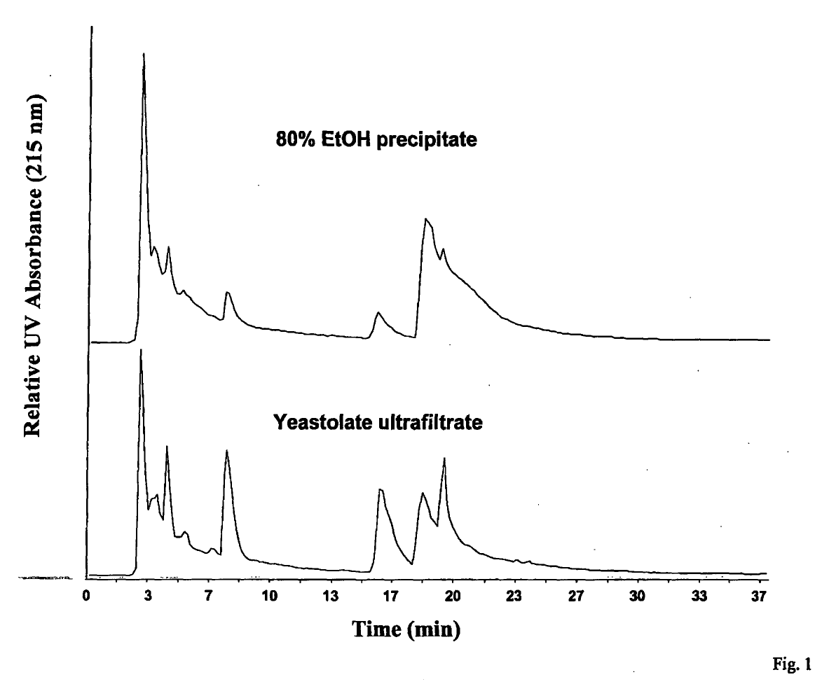

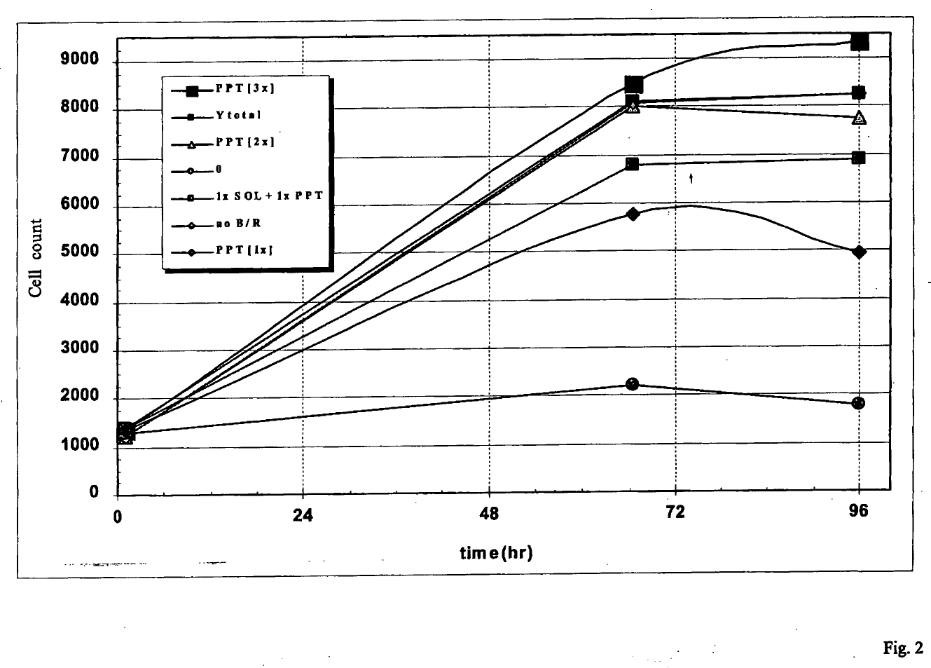

Ethanol (95 vol %/water) was added to the yeastolate ultrafiltrate solution (cat. # 1820048, lot # 1019098 purchased from Gibco/Invitrogen) to a final ethanol concentrations of 60-90 vol %/water. This was undertaken while stirring at room temperature. The supernatant and the precipitate were separated by centrifugation and dried. The dry weight of the precipitate accounted for 3-30% of the total yeastolate ultrafiltrate as listed in Table 1. The HPLC profiles of the yeastolate ultrafiltrate and the 80% EtOH precipitate are shown in FIG. 1. Clearly the peak at 2.8 min is enriched by the 80% EtOH precipitation. FIG. 2 shows the production of GFP (an indicator of protein production) in the presence of the yeastolate ultrafiltrate (positive control), double and triple amount of the 75% EtOH precipitate, and the reconstituted yeastolate from single amount of the 75% EtOH precipitate and the supernatant. Clearly, the precipitate contains the active growth promoting factor(s).

The advantages of the EtOH precipitates are described below:

(A) High Activity.

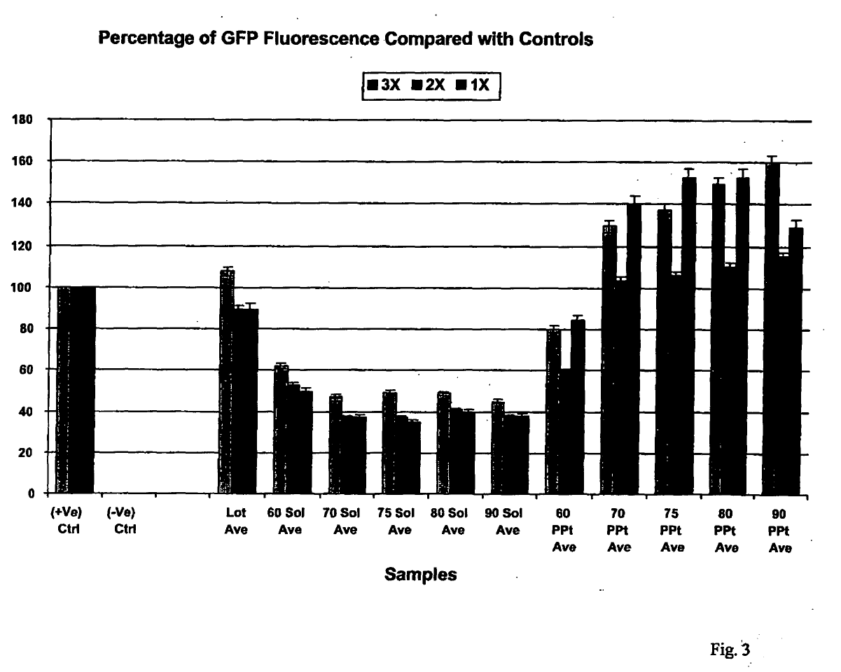

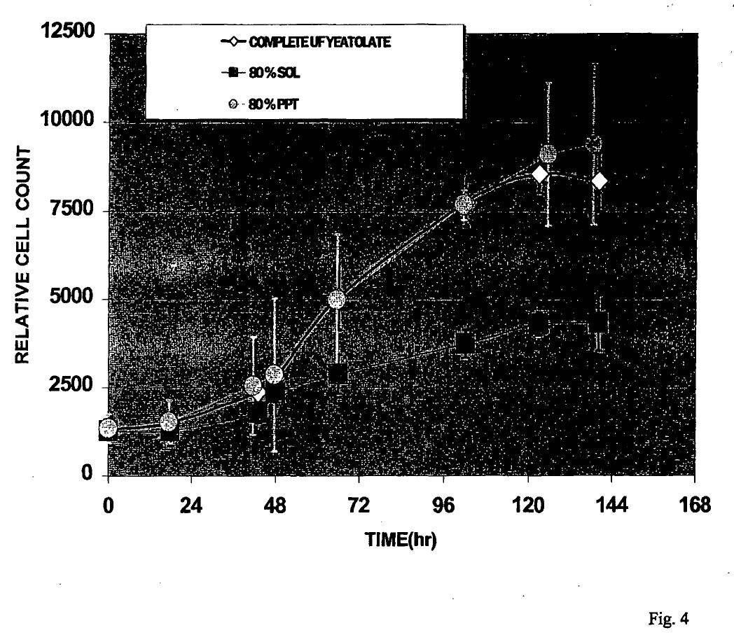

The EtOH precipitates stimulated higher GFP production than the yeastolate ultrafiltrate in 96-well microplates. The reproducibility of the high activity was evaluated by using 6 different lots from a variety of commercially available sources in triplicate. For example, the average and standard deviation of the 80% EtOH precipitates produced from 6 different yeastolate ultrafiltrates and with 3 fold concentration were 156%″ 25% compared to 100″ 36% and 39%″ 70% of the corresponding yeastolate ultrafiltrates and the corresponding supernatants of 80% EtOH precipitation, respectively (FIG. 3). Thus, the most of the active component was precipitated by 80 vol % EtOH/water. This EtOH fractionation of the active component(s) is confirmed in larger scale of using shake flasks (FIG. 4).

(B) Osmotic Pressure in the Culture Medium

70-90% EtOH precipitates approximately 20-50% of the dry weight of yeastolate ultrafiltrates (Table 1). Thus, the same dry weight of 70-90% EtOH precipitates may contain approximately 2-5 times more active component than the yeastolate ultrafiltrates. The major concern of adding higher amount of growth factor medium is the increased osmotic pressure of the culture medium. However, even the addition of triple amount of the 75% EtOH precipitate increased the osmotic pressure in the culture medium to 389 mOsm that is comparable to 378 mOsm measured with the yeastolate ultrafiltrate. Thus, the EtOH precipitation also eliminates the components that increase the osmotic pressure in the culture medium.

(C) Growth Promotion Activity of Yeastolate on the Growth of Human Embryo Kidney Cells 293SF

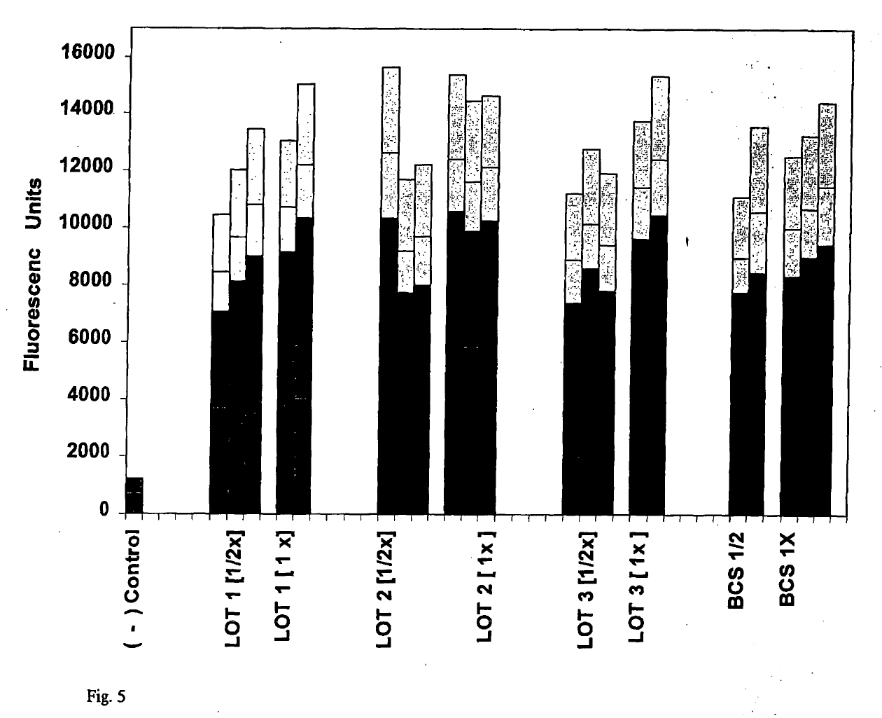

Yeastolate also stimulated the growth of human embryo kidney cells 293SF and enhanced the GFP production in serum free-medium as shown in FIG. 5. Thus, the use of the growth factor(s) in the yeastolate may be extended to mammalian cell cultures.

(D) For fedbatch operations in insect cell cultures using baculovirus expression system decreasing volume of additives will translate in lower dilution of the final product concentration. Also, removing non essential components from yeastolate preparations added to the culture during the fed-batch process will minimize contaminants level in the final bulk product that renders purification simpler.

Characterization of the Component(S) Fractions with Cell Growth and Protein Production Promoting Effects.

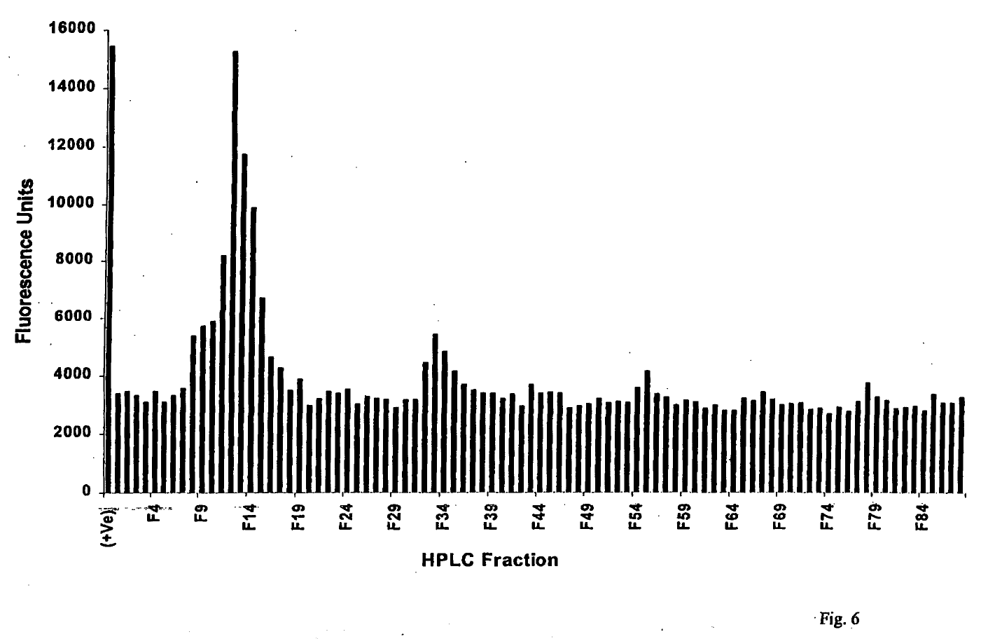

The 80%-EtOH precipitate was fractionated by reverse phase HPLC (250×50 mm C-18 preparative column, water-acetonitrile gradient in the absence of tri-fluoro acetic acid (TFA)). Each fraction was analyzed by their activity of promoting GFP production, resulting the active fractions at 11^{th}-16^{th} fractions (FIG. 6). The active fractions account 30 wt % of the sample injected.

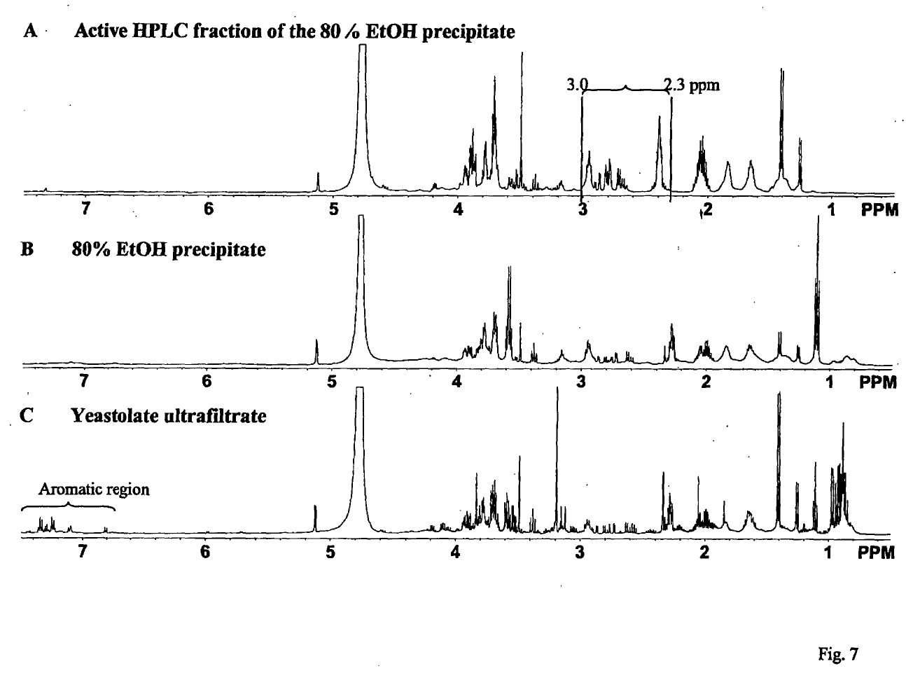

The active fractions 9-17 were pooled, lyophilized and re-fractionated by a reverse phase HPLC (250×22 mm C-18 semi-preparative column, water-acetonitrile gradient in the absence of TFA). Each fraction was analyzed by their activity of promoting GFP production, resulting an active fraction at 6^{th} fraction. The 6^{th} fraction accounts for 50 wt % of the sample injected. FIG. 7 compares the ¹H-NMR spectra of the 6^{th} fraction on HPLC (FIG. 7A), 80% EtOH precipitate (FIG. 7B), and the yeastolate ultrafiltrate FIG. 7C). The enhanced

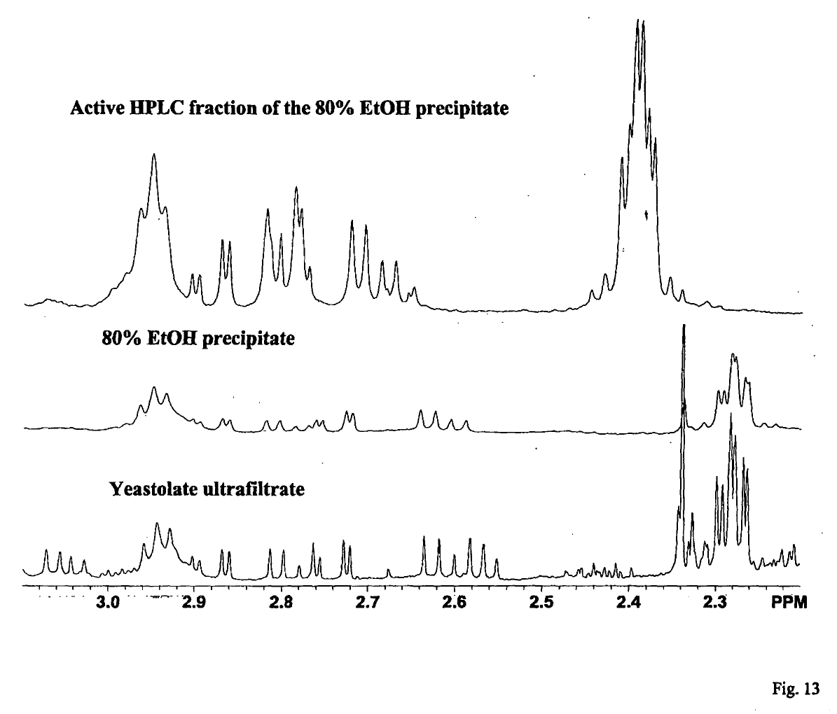

¹HNMR peaks by 80% EtOH precipitation and HPLC purification constitute a doublet of triplets at 2.357 ppm, a doublet of doublets at 2.645 ppm, a doublet of doublets at 2.774 ppm, a doublet at 2.792 ppm and a doublet of doublets at 2.888 ppm as the spectra are expanded in FIG. 13. At least some of them are of the active component. However, some peaks in FIG. 7B that were not enriched by HPLC fractionation may also contribute to the activity.

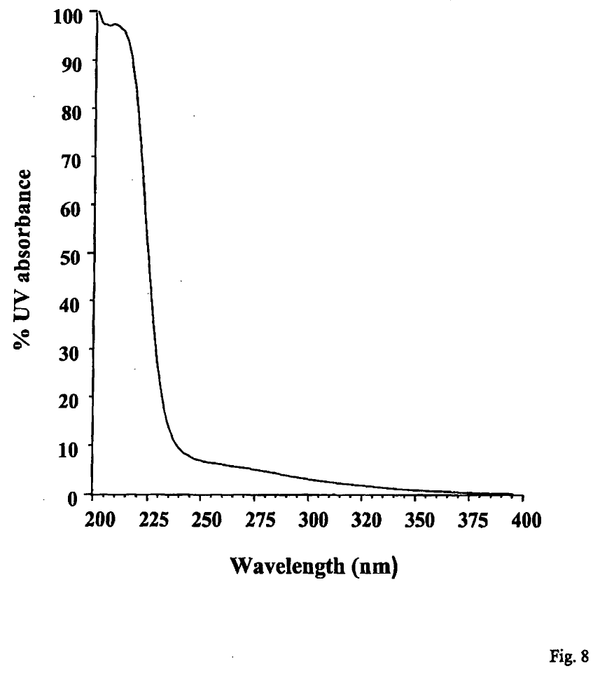

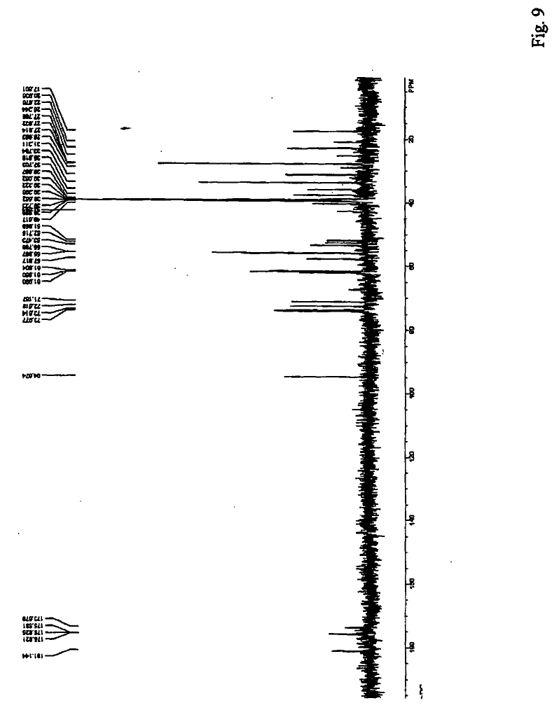

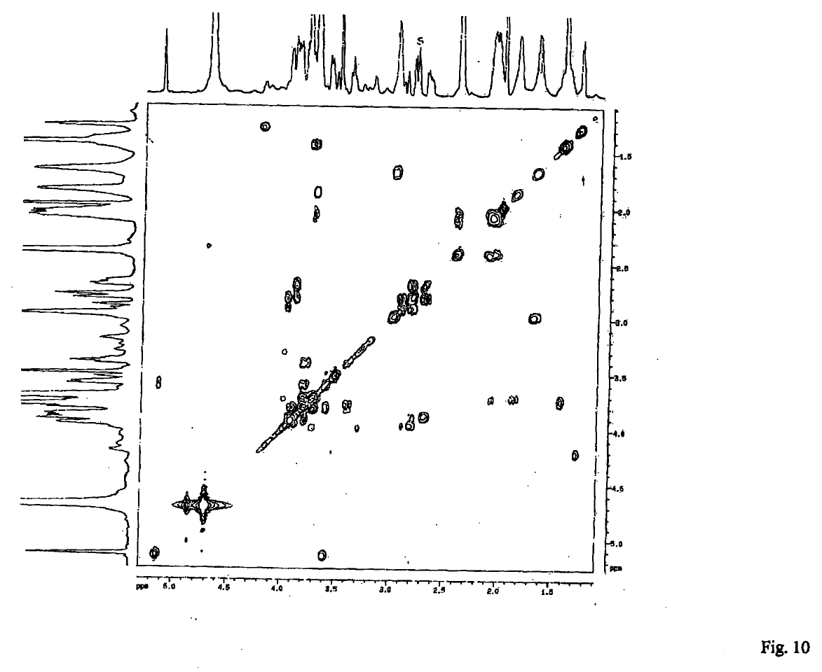

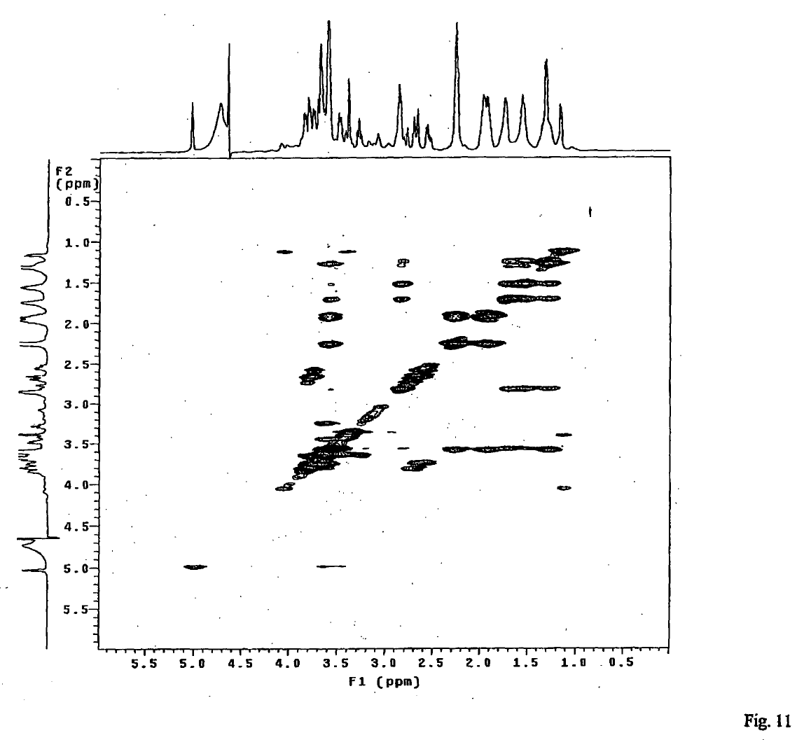

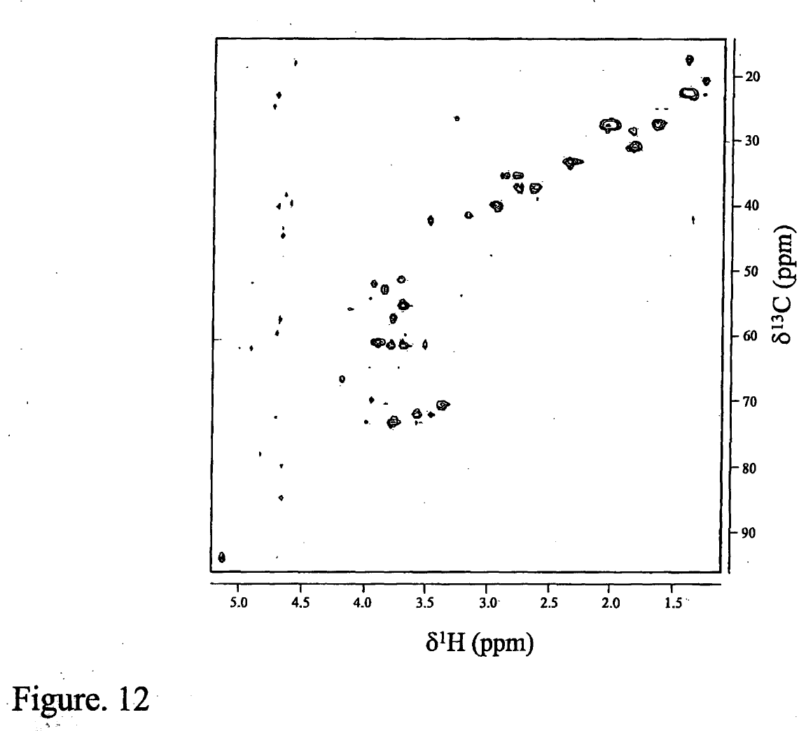

The active 6^{th} fraction was further characterized by the absorption spectrum, ¹H- and ¹³C-NMR (FIGS. 7-9) and their 2D-NMR spectroscopy (FIGS. 10-12). The absorption spectrum shows the absence of aromatic group in the active fraction. The ¹H-NMR spectrum also confirmed it (FIG. 7A). The chemical shifts and the connectivity of the NMR spectra are listed in Table 2.

Development of a Rapid Method to Quantitate the Active Fraction in Each Lot of Yeastolate Ultrafiltrate.

¹H-NMR is used to monitor and quantitate the active fraction in yeastolate ultrafiltrate or 80%-EtOH precipitate. The peaks that are enriched by HPLC fractionation and are isolated from other peaks in FIG. 7A of yeastolate ultrafiltrate are qualified for monitoring. FIG. 13 shows an expanded region of the ¹H-NMR spectrum that contains the enriched peaks. The doublet of triplets at 2.357 ppm, a doublet of doublets at 2.645 ppm, a doublet of doublets at 2.774 ppm, a doublet at 2.792 ppm and a doublet of doublets at 2.888 ppm. may be used to quantify the active component of yeastolate. Thus, ¹H-NMR measurement is a simple, rapid and readily available method for quality control validation of each yeastolate lot.

Activity Evaluation of Fractions from Yeastolate Ultrafiltrate

Materials and Methods

Fractionation of 50× yeastolate ultrafiltrate (YUF, 25 mL at 200 g/L) was carried out through sequential precipitation with different ethanol concentrations at 4° C. The yeastolate precipitated (PPT) under each ethanol concentration range was centrifuged at 3800 rpm for 20 min, collected and freeze-dried. Each fraction was then reconstituted to 100 g PPT/L with Milli-Q water. The activity of each fraction on promoting Sf9 cell growth was evaluated by adding the fraction to cell culture medium (IPL 41) at a concentration of 2 g/L. The response of cell growth on YUF or fraction 1 supplement was examined by adding YUF or F1 to IPL 41 medium to reach a respective concentration of 1, 2, 3 or 4 g/L. Fraction 1 (F1) is the YUF fraction precipitated at a concentration range of 065% ethanol. The cell culture was conducted in 125-ml plastic shake flask with a culture volume of 20 ml. The cell density was examined daily.

Results

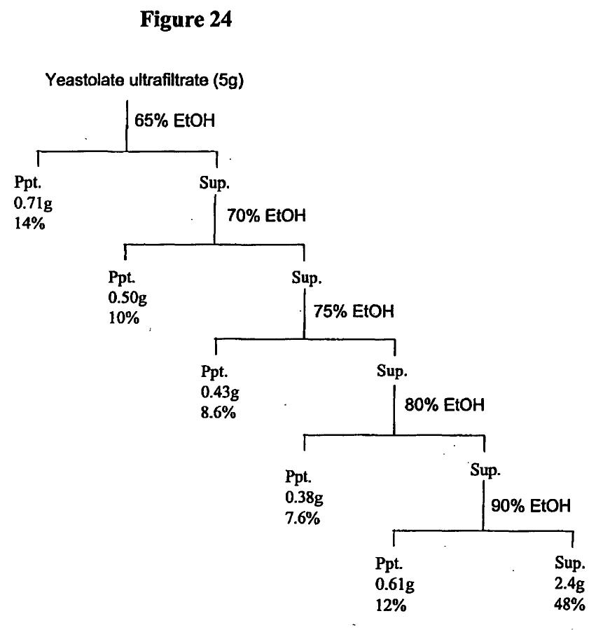

The fractionation results of 50× yeastolate UF by ethanol sequential precipitation are shown in FIG. 24.

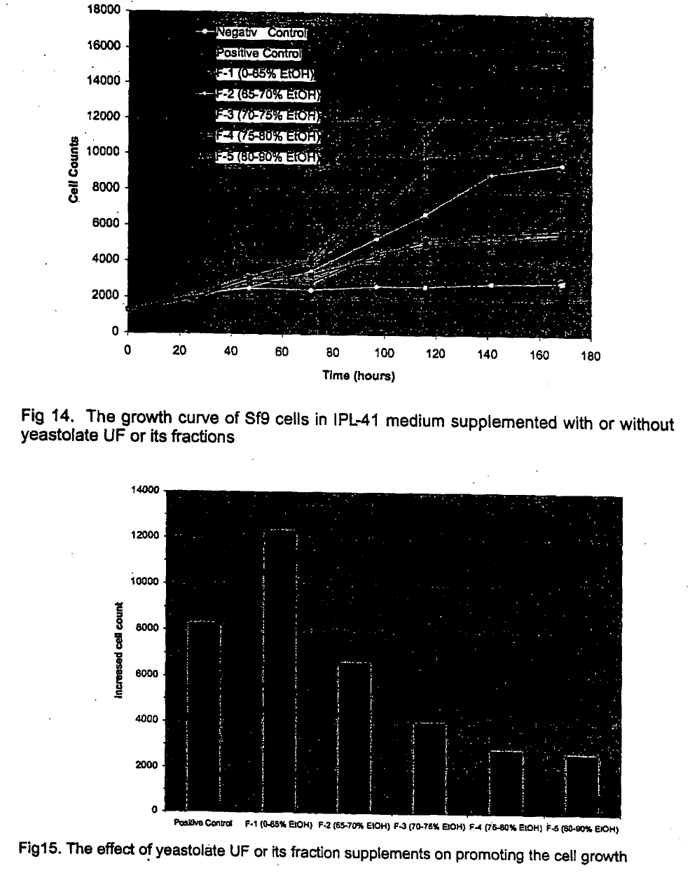

The growth curve of Sf9 cell in IPL 41 medium supplemented with or without YUF or its fraction is presented in FIG. 14. The effect of YUF or its fraction supplements on promoting the cell growth is shown in FIG. 15. The data indicate that F1 has a better activity in promoting Sf9 cell growth. Other fractions precipitated at ethanol concentrations higher than 65% have lower activities than that of YUF (positive control) in promoting Sf9 cell growth.

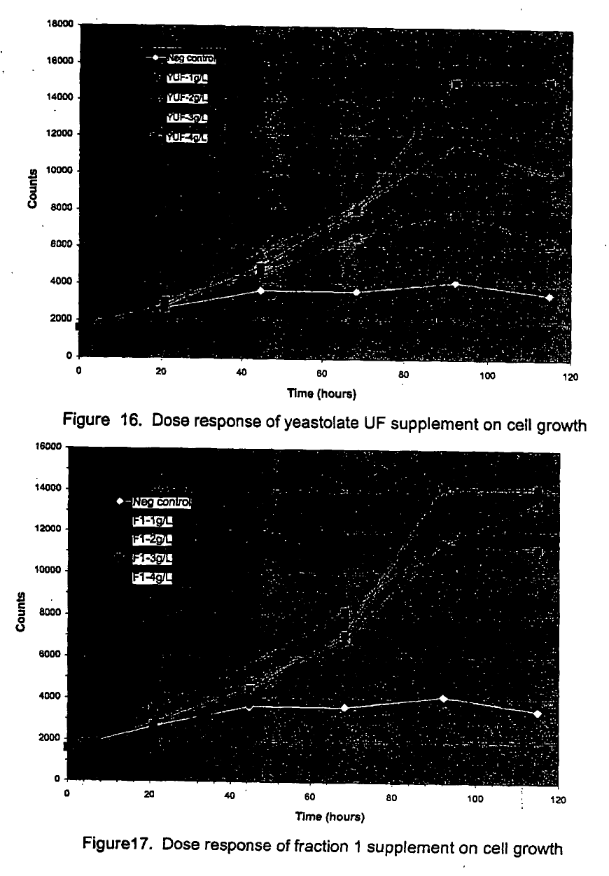

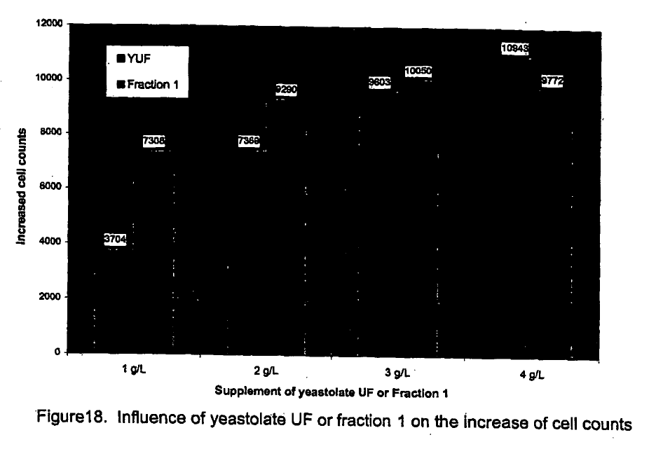

The response of cell growth on the supplementation of different amounts of YUF or F1 to IPL-41 medium is shown in FIGS. 16 and 17. The data in FIG. 16 indicate that the maximum cell count in each sample was increased by the increasing dosage of YUF. The cell count was increased by 3704, 7369, 9603 and 10943 respectively when the IPL 41 medium was respectively supplemented with 1, 2, 3 and 4 g/L of YUF as shown in FIG. 18. Similarly, the cell count of the samples was increased dramatically when F1 was added into the IPL 41 medium. However, similar maximum cell densities (about 14000 counts/ml) were achieved in the samples supplemented with 2, 3 and 4 g/L of F1. These data clearly show that the relationship between the increased cell count and dosage of YUF or F1 supplement is not proportional at higher dosages of YUF or F1. Further increase in the cell density in the samples supplemented with higher dosage of YUF or F1 may be limited by the depletion of other components in the medium. These experimental results also indicate that F1 was more active than YUF in promoting the Sf9 cell growth, and the cell counts increased by the supplement of 1 g/L F1 was nearly double of that achieved by adding 1 g/L of YUF.

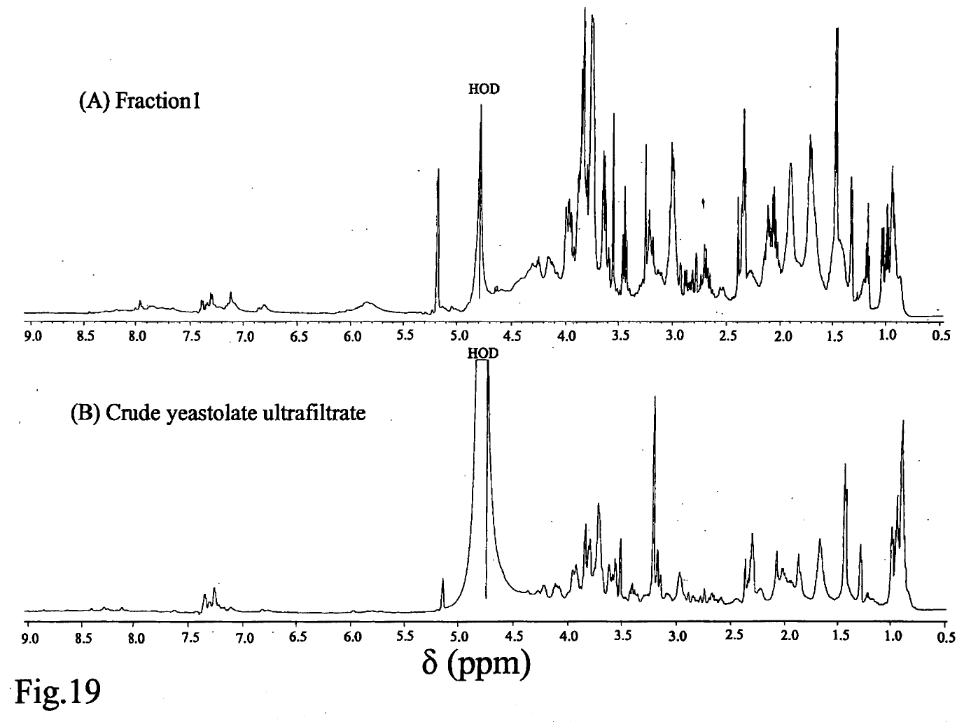

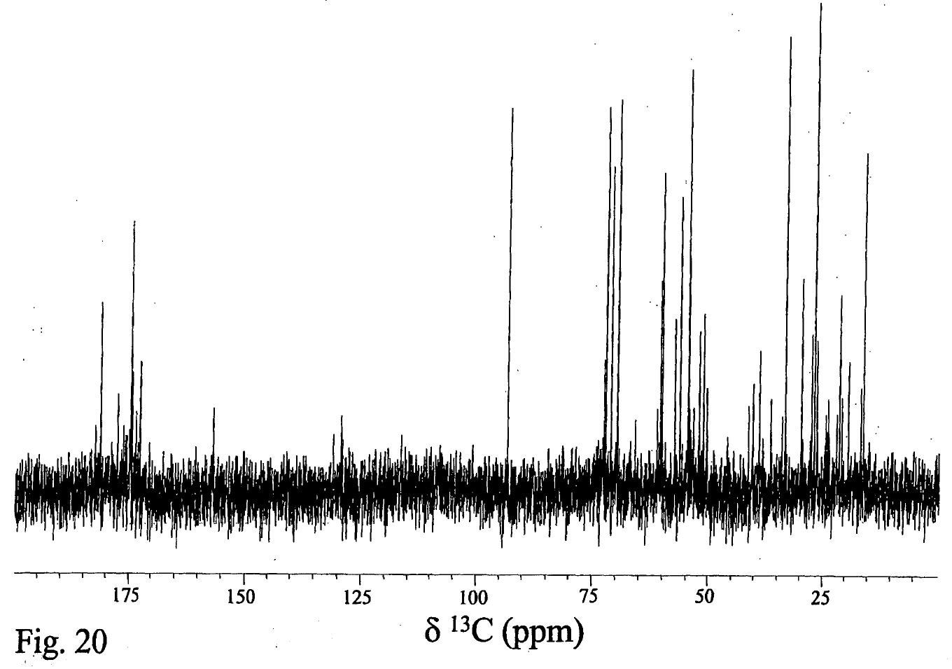

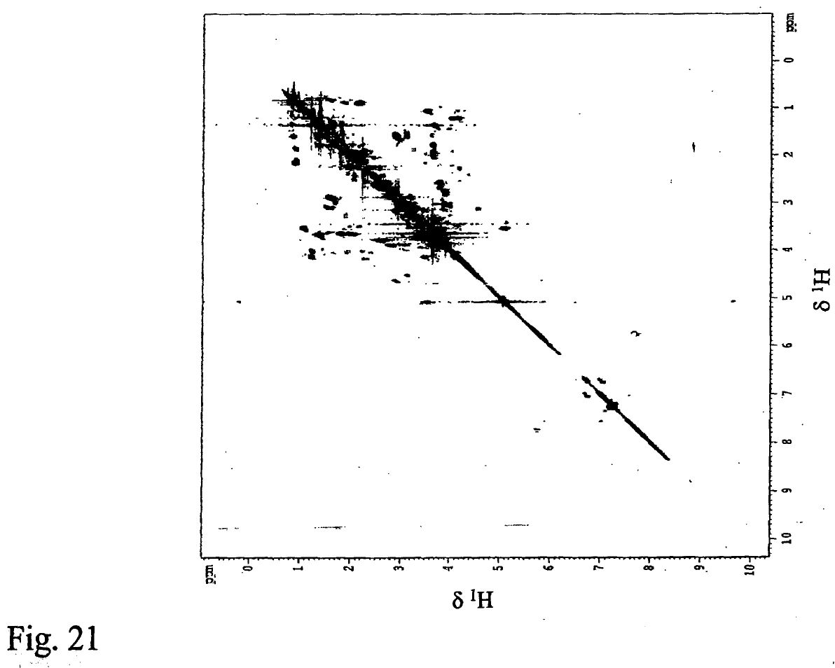

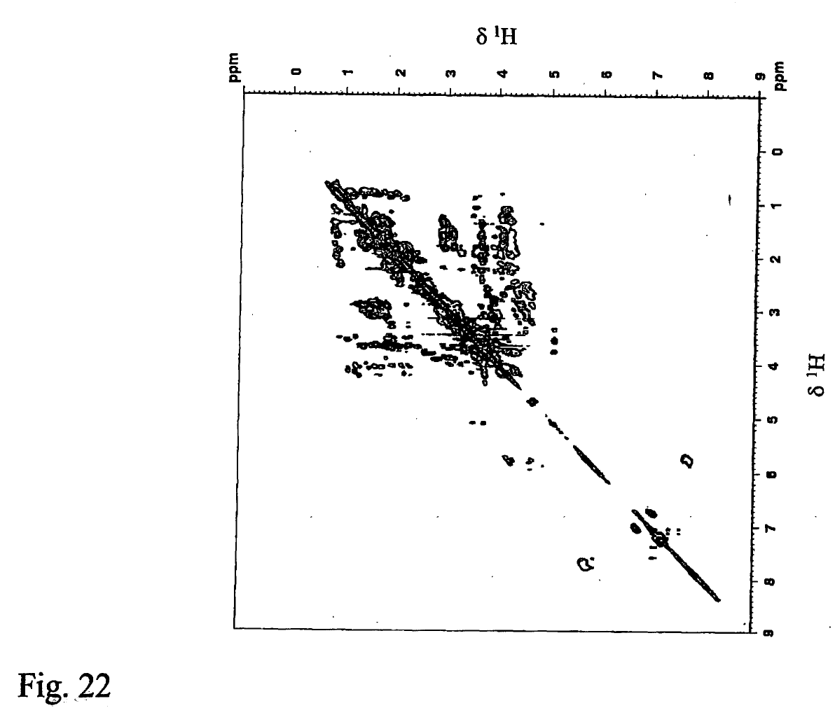

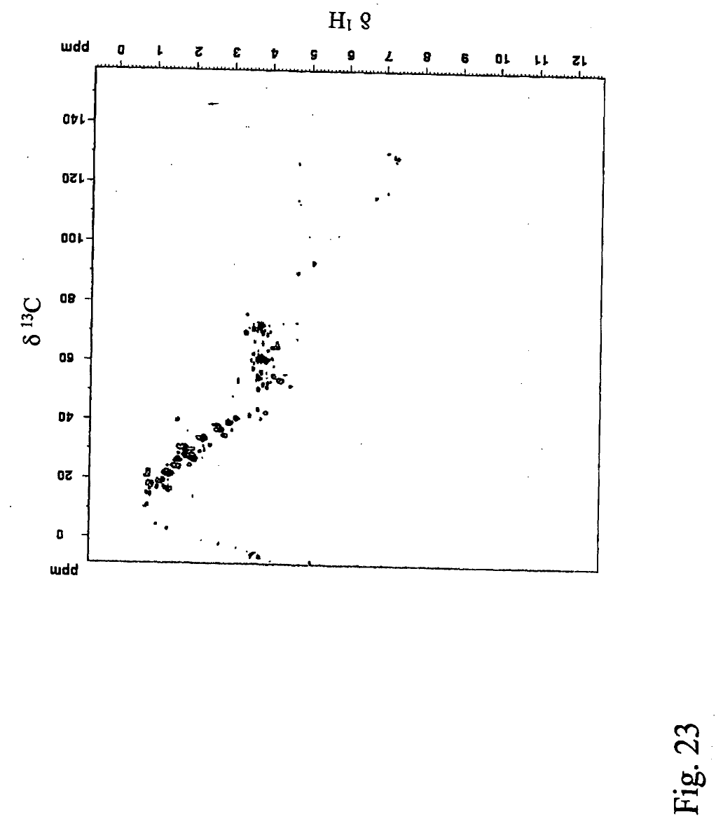

FIGS. 19 to 23 illustrate characteristics of fraction obtained in large scale preparation and demonstrate that the process is essentially scalable. The similarity of FIGS. 19 to 23 to FIGS. 7 and 9 to 12 respectively is striking evidence of the scalable nature of the invention.

Commercial applications of the invention include:

Rapid screening of Saccharomyces cerevisiae (baker's yeast) yeastolate extracts enriched by the active component(s).

Preparation of better defined growth medium from yeastolate ultrafiltrate, leading to better quality control of the protein production by animal cells, such as insect cells.

The low cost of the medium will permit economic mass production of certain type of biopesticides (baculoviruses).

Extension of use of characterized fraction of yeastolate to mammalian cell culture for growth and enhanced expression in replacement of animal derived additives has application in the market of biopharmaceutical manufacturing.

TABLE 1
Yield of ethanol insoluble component of yeastolate at different
concentrations of ethanol (% by weight of yeastolate)

| Ethanol % | Lot 1 | Lot 2 | Lot 3 | Lot 4 | Lot 5 | Lot 6 |
|---|---|---|---|---|---|---|
| 60% | 3.54 | 4.20 | 3.12 | 3.24 | 3.22 | 3.40 |
| 70% | 27.15 | 22.83 | 24.90 | 24.47 | 24.68 | 27.36 |
| 75% | 34.78 | 32.07 | 32.17 | 33.22 | 30.73 | 35.12 |
| 80% | 41.20 | 42.63 | 37.51 | 37.73 | 40.62 | 44.32 |
| 90% | 50.99 | | 49.39 | 49.49 | 51.60 | 53.79 |

TABLE 2
Peaks in H-NMR and C-13 NMR spectra of the active HPLC fraction of the 80% EtOH precipitate^{a)}

| | Carbon | | Connected proton | |
|---|---|---|---|---|
| Connectivities | Chemical shift (ppm) | Multiplicity (Dept) | Chemical shift | Proton |
| 1A | 94.154 | CH | (5.125, 5.117) | Doublet |
| 3A | 73.466 | CH | 3.769 | |
| | 73.092 | CH | 3.751 | |
| 2A | 71.991 | CH | (3.586, 3.579, 3.566, 3.559) | Doublet of doublet |
| | 70.646 | CH | (3.395, 3.375, 3.357) | Triplet |
| | 61.476 | CH | 3.683/3.800 | |
| 4A* | 61.084 | CH | (3.908, 3.900)/(3.890, 3.854) | Doublets |
| | 57.306 | CH | 3.780 | |
| 2F | 55.502 | CH | 3.682 | |
| 4A* | 55.427 | CH | 3.706 | |
| | 55.362 | CH | 3.695 | |
| 2D | 52.951 | CH | (3.854, 3.848, 3.837, 3.831) | Doublet of doublet |
| 2E | 52.198 | CH | 3.933 | |
| | 51.435 | CH | 3.717 | |
| | 42.344 | CH | 2.357 | Singlet |
| 2C | 39.988 | CH | (2.970, 2.955, 2.940) | Triplet |
| 1D | 37.178 | CH | (2.795, 2.788, 2.760, 2.753)/(2.670, 2.654, 2.635, 2.618) | 2 Doublet of doublet |
| 1E | 35.392 | CH | (2.907, 2.899, 2.874, 2.866)/(2.810, 2.777) | Doublet/doublet of doublet |
| 1B | 33.239 | CH | (2.375, 2.370, 2.360, 2.355, 2.354, 2.340) | Doublet of triplet |
| 1F | 30.780 | CH | 1.841 | Multiplet |
| 2B | 27.393 | CH | 2.07 | |
| 1C | 27.312 | CH | (1.687, 1.671, 1.656, 1.641, 1.626) | Pentuplet |
| | 22.336 | CH | 1.408 | Multiplet |
| | 20.42? | CH | 1.416, 1.401 | Doublet |
| 5A | 17.078 | CH | 1.266, 1.253 | Doublet |
| ^{a)}Carbon-carbon and carbon-proton connectivities of the component(s) of the active HPLC fraction of 80% EtOHl precipitate derived from C-13 NMR and H-NMR and proton-proton and carbon-proton 2D NMR experiments. Chemical shift values in parenthesis represent splitting of a protons. Chemical shift values separated by a slash represent shifts of different protons attached to the same carbon atom. Carbon atoms connected to each other are indicated by a letter | | | | |
| # (e.g. A), and the order of connectivity is indicated by a number series (e.g. 1, 2, 3 . . .) | | | | |

1. A process for preparing an active animal cell-growth-enhancing fraction of a hydrolysate of microorganism ultrafiltrate material which process comprises forming a precipitate of said fraction from an aqueous solution of said hydrolysate of said if microorganism ultrafiltrate with a water-miscible solvent.

2. The process of claim 1 wherein the hydrolysate of microorganism ultrafiltrate material comprises a yeastolate ultrafiltrate.

3. The process of claim 1 additionally comprising separating and drying said precipitate.

4. The process of claim 1 additionally comprising fractionating said precipitate and selecting high activity fractions therefrom.

5. The process of claim 1 wherein said water-miscible organic solvent is selected from the group consisting of alkanols, alkyl sulfoxides, ketones and alkyl nitrites.

6. The process of claim 4 wherein said water-miscible solvent is selected from the group consisting of an alcohol of the formula CH₃—(CH₂)ₙ—OH (n=1 to 4), an alkyl sulfoxide of the formula CH₃—(CH₂)ₙ—SO—(CH₂)ₘ—CH (n=0 to 2, m=0 to 2), an alkyl nitrile of the formula CH₃—(CH₂)ₙ—CN (n=0 to 2) and a ketone of the formula CH₃—(CH₂)ₙ—CO—(CH₂)ₘ—CH₃ (n=0 to 2, m=0 to 2)

7. The process of claim 5 wherein said water-miscible solvent is a C₁-C₅ alcohol.

8. The process of claim 7 wherein said water-miscible solvent is ethanol.

9. The process of claim 8 wherein said precipitate is formed with an ethanol concentration of about 65%.

10. An active animal cell-growth-enhancing fraction of a plant tissue, animal tissue or microorganism ultrafiltrate material substantially free of aromatic group bearing compounds prepared according to the process of claim 1.
